(19) United States
(12) Patent Application Publication
(10) Pub. No.: US 2004/0019022 A1
(43) Pub. Date: Jan. 29, 2004

(54) Use of 2,3 alkylcarbonyloxybenzoic acids, derivatives and analogues therefrom in the treatment of tissue and cellular dysfunction, damage and injury in mammals

(76) Inventors: Karen Stec, Barrington, IL (US); Israel Rubinstein, Highland Park, IL (US); David Eiznhamer, Bloomingdale, IL (US); Ze-Qu Xu, Woodridge, IL (US); Michael Flavin, Darien, IL (US)

Correspondence Address: Jeffrey M. Hoster, 13 Woodland Drive, Lemont, IL, 60439, US

(21) Appl. No.: 10/622,302

(22) Filed: Jul. 18, 2003

Related U.S. Application Data
(60) Provisional application No. 60/398,523, filed on Jul. 25, 2002.

(51) Int. Cl. A61K 38/12; A61K 38/00; A61K 38/16; A61K 31/704; A61K 31/60; A61K 31/496

(52) U.S. Cl. 514/159; 514/8; 514/192; 514/34; 514/252.13; 514/29; 514/2; 514/253.08; 514/312

(57) ABSTRACT

A method for treating cellular and tissue damage is disclosed. The inventive method comprises the use of 2,3-alkylcarbonyloxybenzoic acid and salts thereof in the prevention and treatment of dysfunction, damage, and/or injuries to organs, tissues and/or cells in human or animal subjects caused by diseases, infections and conditions such as pneumonia, coronavirus, multiple transfusions, trauma, ischemic-reperfusion dysfunctions, stroke, drug overdose, and severe acute respiratory syndrome. The 2,3-alkylcarbonyloxybenzoic acid may be used alone or in combination with other therapeutic agents such as antibiotics. The acid may be administered in any practical delivery form, and in free acid or buffered form.

### RELATED APPLICATIONS

This application claims benefit of U.S. provisional Application Ser. No. 60/398,523, filed Jul. 25, 2002.

### FIELD OF THE INVENTION

This invention is directed to the field of preventing and treating organ, tissue, and cellular dysfunction, damage and injury. Such dysfunction, damage or injury can result from diseases, infections, or conditions which result in the formation of reactive oxygen species, or the inflammatory and immunological responses to such diseases, infections, or conditions.

Many diseases, infections, and conditions cause dysfunction, damage, and injury to mammalian systems through multiple mechanisms. Injuries such as ischemic stroke cause primary damage through the blockage of oxygen-rich blood flow to a portion of the brain. Many other similar injuries result from the reperfusion of tissue following the impaired oxygen flow. A typical mechanism for such injury begins with the interruption of blood flow to an area of a mammalian body such as the brain, heart, muscle, skin, bone or other organ. The loss of blood flow results in lack of oxygen, and the depletion of high energy molecules such as ATP. Further injury occurs as a result of the inability to transport toxic metabolites remaining in the cells via the systemic circulation.

Often in these diseases, infections, or conditions, excessive amounts of free radicals are produced. This free radical production can be catalyzed by free iron present in the cell. Indeed in many of these diseases, infections, or conditions excess free iron is present in the cell. While mammalian cells typically have an array of defenses to combat free radical damage, in many diseases, infections, or conditions, it is speculated that either these defenses are inhibited or free radical production exceeds the capability of such defenses.

Free radical damage is especially a problem following events resulting in ischemia. In addition to the primary injuries described above, there is conversion of xanthine dehydrogenase to xanthine oxidase in the injured areas. A typical therapy for conditions such as stroke, myocardial infarction and other ischemias is the administration of oxygen in an attempt to cause the resumption of blood flow and limit the damage caused by the lack of adequate tissue perfusion. In the presence of xanthine oxidase, however, this reperfusion results in the production of reactive oxygen species and resulting secondary injuries.

In yet other cases, bacterial or viral infections cause dysfunction, damage, and injury to organs, tissues, and cells. Components of bacteria or viruses, such as lipopolysaccharide (LPS) from gram-negative bacteria (endotoxin), play a major role in initiating the inflammatory processes that result in dysfunction, damage, and injury to organs, tissues, and cells. One example of such a condition is the recent worldwide outbreak of Severe Acute Respiratory Syndrome (SARS) which may be caused by a novel coronavirus. Patients with SARS often succumb to progressive respiratory failure as a result of diffuse alveolar damage. This damage is most likely mediated through the molecular mechanisms discussed above.

Still other diseases, infections, or conditions cause organ, tissue, or cellular damage through other mechanisms, or through mechanisms that are yet to be understood.

Thus, there is a need to prevent or reduce the damage caused by diseases, infections, or conditions described above. The present invention details a series of compounds and method of application which result in such an effect.

### SUMMARY OF THE INVENTION

The invention encompasses methods for preventing and/or treating dysfunctions, damages, and/or injuries resulting from an excess of reactive oxygen species, and the inflammatory and immunological responses to assaults caused by physiological disorders, which include, but are not limited to, such dysfunctions resulting from ischemia and/or reperfusion, by the use of 2,3-alkylcarbonyloxybenzoic acids in which the alkylcarbonyloxy group has 2-18 carbon atoms. The structure of such 2,3-alkylcarbonyloxybenzoic acids is preferably:

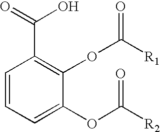

where R₁ and R₂ are selected from the group consisting of straight chain, branched, alkyl groups, and wherein such alkyl groups can be the same or different, each consisting of 1-17 carbon atoms.

The invention further encompasses the prevention and/or treatment of dysfunction, damage, and/or injuries resulting from excessive levels of toxic levels of metal ions, toxins, or infectious pathogens by use of the same acids.

One particularly preferred compound is 2,3-diacetoxybenzoic acid. The preferred method of prevention and/or treatment involves administering 2,3-alkylcarbonyloxybenzoic acid to a subject known or suspected to have suffered dysfunction, damage, and/or injuries of the nature described above. The 2,3-alkylcarbonyloxybenzoic acid can be administered by any known therapeutic method, and in combination with other compounds known or believed to prevent or treat the diseases, infections, or conditions responsible for the dysfunction, damage, and/or injuries described above. The 2,3-alkylcarbonyloxybenzoic acid can be administered as the free acid, with any acceptable delivery system, or in salt form. More than one of the 2,3-alkylcarbonyloxybenzoic acid compounds can also be blended.

### DETAILED DESCRIPTION OF THE PREFERRED EMBODIMENTS

In a preferred embodiment, a therapeutically effective amount of a compound selected from the group consisting of 2,3-alkylcarbonyloxybenzoic acids and salts thereof in which the alkylcarbonyloxy group has 2-18 carbon atoms is administered to subjects who have indications of organ, tissue and/or cellular dysfunction, damage and/or injury. For purposes of this application, any reference to 2,3-alkylcarbonyloxybenzoic acids shall be construed to include the salts thereof. Compounds of the group consisting of 2,3-alkylcarbonyloxybenzoic acids have been shown to ameliorate the damage resulting from increased vascular permeability and the resulting pulmonary edema caused by increased microvascular permeability to protein and solutes resulting from Adult Respiratory Distress Syndrome and sepsis. This effect is detailed in U.S. Pat. No. 5,504,111, issued Apr. 2, 1996 to Flavin, et al. Additionally, these compounds are now believed to be useful in the treatment of individuals suffering from inhalation anthrax. This effect is detailed in U.S. patent application Ser. No. 10/284,768, filed on Jul. 25, 2002.

While not wishing to be bound by theory, it appears that the class of 2,3-alkylcarbonyloxybenzoic acid compounds possesses characteristics especially suitable for the repair of organ, tissue, and/or cellular dysfunction, damage and/or injury. It is believed that, upon delivery to normal organs, tissues, and cells, these compounds have no effect; including a lack of effect on normal levels of beneficial anti-oxidants. The administration of these compounds would not perturb the pro/anti-oxidant balance in a normal organ, tissue, or cell with basal levels of free radicals and antioxidant mechanisms. In the diseases, infections, or conditions indicated, there is a perturbation of these basal levels resulting in increases in damaging free radicals. The 2,3-alkylcarbonyloxybenzoic acid compounds may act to ameliorate the damage caused by this excess production. Indeed, administration of these compounds prior to the free radical-producing insult should result in a protective effect, potentially preventing the dysfunction, damage, and/or injury. However, upon delivery to cells having injury or other signs of perturbation, the compounds provide a protective effect with regard to damaged organs, tissues and cells.

A particularly preferred compound in the class of 2,3-alkylcarbonyloxybenzoic acid compounds is 2,3-diacetoxybenzoic acid. In an especially preferred embodiment, 2,3-diacetoxybenzoic acid is administered to a subject, in a therapeutically effective amount, through any means effective to make such delivery. The diseases, infections or conditions to be treated by administration of the 2,3-alkylcarbonyloxybenzoic acid include, but are not limited to: acute myocardial syndrome, myocardial infarction, ischemic stroke, reperfusion following surgery, gastric mucosal injury (ulcer), hepatic microcirculatory dysfunction (hepatitis and alcoholic liver disease), systemic shock of all types, pancreatic microcirculation disturbances in diabetes, ischemia/reperfusion injury of skeletal muscle caused by thromboembolic events or trauma, ischemia/reperfusion crisis in organ transplant recipients, Duchene's muscular dystrophy, prevention of paraplegia secondary to spinal cord injury, cerebrovascular insufficiency, atherosclerosis, vascular ophthalmopathies, sinusitis, cystic fibrosis, rhinitis, decubitus ulcer, peripheral vascular insufficiency, renal insufficiency, ischemic bowel disease, asthma, chronic obstructive pulmonary disease, pneumonia, bronchitis, pulmonary colonization with bacteria, such as Haemophilus influenzae and Pseudomonas aeruginosa, acne, rosacea, alpha one-antitrypsin deficiency, pulmonary edema, pneumonic plague, congestive heart failure, pulmonary hypertension, lymphangitis, arthritis, bums, diverticulitis, diverticuloisis, lupus erythematosus, rheumatoid arthritis, ankylosing spondylitis, pemphigus vulgaris, pemphigoid, Stevens-Johnson syndrome, drug-induced skin reactions, psoriasis, Alzheimer's disease, multiple sclerosis, eclampsia, pre-eclampsia, malignant hypertension, hypertensive crisis, encephalopathy, encephalitis, meningitis, neuritis, prion-related diseases, systemic hypertension, inflammatory bowel disease, cirrhosis, hepatic encephalopathy, inhalation lung injury, infant respiratory distress syndrome, acute respiratory distress syndrome, severe acute respiratory syndrome, aphtous stomatitis, stomatitis, esophagitis, duodenitis, adenitis, salivary gland inflammation, gingivitis, periodontitis, caries, vaginitis, Parkinson's disease, Huntington's disease, hepatitis, AIDS, Lyme's disease, Rickettsiosis, sarcoidosis, idiopathic pulmonary fibrosis, interstitial lung disease, emphysema, bronchiestasis, a typical mycobacteria, fungal infections, viral infections such as coronavirus, respiratory synctyial virus, metapneumovirus, rhinoviruses, paramyxoviruses, herpes, adenovirus, Epstein Barr virus, parainfluenza viruses, and human immunodeficiency virus, bacterial infections such as Mycoplasma pneumoniae, Chlamydia pneumoniae, Streptococcus pneumoniae, Klebsiella pneumoniae, Staphylococcus aureus, Acinetobacter, Streptococci, Enterococci, Eschericha coli, Mycobacterium tuberculosis and Mycobacterium avium, lichen planus, conjunctivitis, conjunctivitis sicca, nickel, lead, cobalt and other heavy metal poisoning, including accumulation of trace elements and hemochromatosis.

It is expected that application of the 2,3-alkylcarbonyloxybenzoic acid will be especially beneficial for certain classes of dysfunction, damage or injuries. These include ischemia-reperfusion dysfunctions, severe acute respiratory syndrome (SARS), and hemochromatosis. The amelioration of pulmonary edema caused by the demonstrated reduction in microvascular permeability would be especially beneficial to patients suffering from severe acute respiratory syndrome.

The therapeutic application of the 2,3-alkylcarbonyloxybenzoic acid is as follows. A condition, such as an injury or disease, is identified in a subject that is causing, or is expected to cause, organ, tissue, or cellular damage. Such damage can be the primary result of the injury or disease, or it can be a secondary result of healing or therapy. The 2,3-alkylcarbonyloxybenzoic acid can be applied by any appropriate administration means, and can be applied for either preventative or therapeutic use.

Such administration means include, but are not limited to, oral, intravenous, topical, cutaneous, transdermal, subcutaneous, intramuscular, inhalation, intranasal, rectal, vaginal, urethral, ocular, sublingual, transpulmonary, intraperitoneal, mucosal, transmucosal, and irrigation administration. The 2,3-alkylcarbonyloxybenzoic acid compounds may be encapsulated, tableted or incorporated into an emulsion syrup (either oil-in-water or water-in-oil) for oral administration. The dosage forms can include pills, powders, granules, elixirs, tinctures, or suspensions. One specific form of powder which can be used is a lyophilized powder which can be reconstituted with diluents for parenteral injections. The formulations may utilize carriers conventionally used in the formulation of pharmaceuticals, including but not limited to starches, lactose, calcium sulfate dihydrate, terra alba, croscarmellose sodium, carboxymethylcellulose and salts thereof, magnesium stearate or stearic acid, talc, pectin, gum acacia, xanthan gum, gellan gum, agar, gelatin, maltodextrins, microcrystalline celluose, and colloidal silicon dioxide.

The amount is preferably, but not exclusively, in the range of 0.1-100 mg/kg of body weight of the subject. More preferably, the range is 5-50 mg/kg of body weight, and most preferably, the range is 5-20 mg/kg of body weight. While preferred, these ranges are exemplary only. Combinations with other therapeutic agents, the delivery system, and the type and stage of the disease or condition may result in use of amounts outside the above-described ranges. The dosages described above can be administered over the period of time necessary to show effectiveness, and can be administered as single dose, multiple doses, or as a continuous or intermittent infusion over time. Sustained release agents such as glyceryl monostearate, glyceryl distearate, polyethylene glycol (preferred molecular weight: 400-10000) may be used. Wax may be included in such sustained release formulations.

While not wishing to be bound by theory, it is believed that, following administration to a subject, at least a percentage of 2,3-diacetoxybenzoic acid is converted to 2,3-dihydroxybenzoic acid, which has therapeutic effectiveness. Thus, all or a portion of a treatment product may also comprise 2,3-dihydroxybenzoic acid, whether administered directly or through chemical conversion in the subject.

In one embodiment, the 2,3-alkylcarbonyloxybenzoic acid is produced in solid form. In preparing the therapeutic product, the solid form may be formulated into a liquid form by use of a sodium bicarbonate buffer present in an amount to effectively solubilize and stabilize the acid. One skilled in the art will recognize that the production of the acid and subsequent preparation of the therapeutic product may take any conventional form used for drugs having similar physical properties. For example, organic solvent evaporation, supercritical fluid extraction, and other conventional methods of preparation may be used.

Depending on the particular use, the product may remain in the free acid form, or a buffered salt may be formed. If a buffered salt is formed, it can then be constituted into aerosol form using conventional methods of forming aerosol products.

Treatment with 2,3-alkylcarbonyloxybenzoic acid can be used as a sole treatment mechanism, or in combination with other therapeutic agents. For example, 2,3-alkylcarbonyloxybenzoic acid can be administered in conjunction with antibiotic therapy. For certain therapeutic uses, for example, the combination of 2,3-alkylcarbonyloxybenzoic acid and one or more antibiotics is expected to be an especially useful therapeutic combination. The antibiotics may be selected from the group comprising fluoroquinolines, doxycycline, rifampin, vancomycin, imipenem, chloramphenicol, penicillin, clindamycin, clarithromycin, gentamicin, beta-lactam antibiotics, ketolides, peptide antibiotics, anti-fungal agents, quinupristin/dalfopristin, linezolid and analogs, homologs, and derivatives thereof which have antibiotic functionality. The combination of 2,3-alkylcarbonyloxybenzoic acid and therapeutic agents for the prevention and treatment of blood clots, strokes, hypertension and myocardial infarction are also expected to be a useful therapeutic combination. Examples of such therapeutic agents include, but are not limited to thrombolytic agents, tissue plasminogen activators, and platelet inhibitors. Examples of such agents include, but are not limited to alteplase, tenecteplase, anistreplase, reteplase, streptokinase, urokinase, dipyridamole, and clopidogrel.

2,3-alkylcarbonyloxybenzoic acid can also be combined with other agents which are used for therapeutic, cosmetic and diagnostic purposes. Agents of particular interest include but are not limited to corticosteroids, mineralosteroids, non-steroidal anti-inflammatory drugs, beta-agonists including dopamine, immunomodulators such as colchicine and macrolides, prolastin, drotrecogin alfa, penicillamine, desferroxamine, vitamins, antiviral agents such as ribavirin, nucleoside analogues, nonnucleoside inhibitors, protease inhibitors, fusion inhibitors, and antifungal drugs such as ketoconazole, fluconazole, and itraconazole, including analogues thereof.

The following example details the potential therapeutic effect of 2,3-alkylcarbonyloxybenzoic acids.

### EXAMPLE 1

### Administration of 2,3-Diacetoxybenzoic Acid to Guinea Pigs

The potential therapeutic effect of 2,3-alkylcarbonyloxybenzoic acids is exemplified by data generated in the study of 2,3-diacetoxybenzoic acid in guinea pigs. 2,3-diacetoxybenzoic acid was administered via intra-peritoneal injection to guinea pigs administered aerosolized LPS. Each animal was treated with 150 mg/kg of 2,3-diacetoxybenzoic acid or vehicle 1 hour prior to and 4 hours following LPS administration. Twenty four hours following administration of LPS, bronchoalveolar lavage (BAL) was performed. BAL fluid total cell count, neutrophil count, and albumin concentration were determined; the results are shown in Table 1. Intra-peritoneal administration of 2,3-diacetoxybenzoic acid appeared to have no effect on LPS-induced neutrophil recruitment into the lung. However, 2,3-diacetoxybenzoic acid treatment did produce a statistically significant reduction in albumin leakage (44%), a component of LPS-induced edema formation indicating a decrease in lung vascular permeability.

TABLE 1
BAL fluid cell counts in guinea pigs

| | | Total Cell | Neutrophil | Albumin | Lung |
|---|---|---|---|---|---|
| Admi- | Dose^{b} | (×10⁶/mL | (×10⁶/mL | (μg/mL | Wet/Dry |
| nistered | (mg/kg) | BALF) | BALF) | BALF) | Wt. (g) |
| LPS + | NA | 93 ± 14^{a} | 70 ± 8 | 1078 ± 178 | 4.06 ± 0.08 |
| vehicle | | | | | |
| (n = 5) | | | | | |
| LPS + | 150 | 112 ± 14 | 80 ± 11 | 605 ± 78* | 4.38 ± 0.08* |
| DABA | | | | | |
| (n = 5) | | | | | |

BALF-Bronchoalveolar lavage fluid
^{a}mean ± SEM
^{b}DABA or vehicle were administered 1 hour prior to and 4 hours post LPS exposure
*indicates statistically significant (p < 0.05) difference compared to vehicle-treated group

An awake rat model of cecal ligation and perforation (CLP)-induced sepsis was employed to examine the therapeutic effect of 2,3-diacetoxybenzoic acid in sepsis induced acute lung injury. Oxidative stress resulting in increased lung permeability is characteristic of the onset and progression of acute lung injury in this model. As shown in Table 2, lung microvascular permeability increases were reduced in animals treated with 2.5 mg/kg/h of 2,3-diacetoxybenzoic acid as a continuous infusion beginning one hour and ending 24 hours following the induction of sepsis. 2,3-Diacetoxybenzoic acid treated animals appeared to have improvement in sepsis-associated symptoms such as lethargy, diarrhea, anorexia, and abdomenal distention. Finally, data suggests that 2,3-diacetoxybenzoic acid normalized body temperature, respiratory rate, and cardiac output in these animals.

TABLE 2
Cecal Ligation and Perforation in Rat

| | | |
|---|---|---|
| Systemic WBC | BAL WBC | BAL protein |
| (×10⁹/liter) | (×10⁹/liter) | (mg/dl) |

| | 1 hour | 24 hours | 24 hours | 24 hours |
|---|---|---|---|---|
| SHAM | 6.0 ± 1.0^{a} | 9.5 ± 2.2 | 0.10 ± 0.05 | 1.25 ± 0.91 |
| CLP | 5.5 ± 1.0 | 2.7 ± 0.9 | 0.21 ± 0.13 | 3.04 ± 1.86 |
| CLP-DABA | 6.7 ± 1.1 | 3.6 ± 1.4 | 0.04 ± 0.02* | 1.45 ± 1.73 |

^{a}mean ± SEM
*indicates statistically significant (p < 0.05) difference compared to vehicle-treated group

What is claimed is:

1. A method for preventing and/or treating dysfunction, damage and/or injuries to organs, tissues, and/or cells in human or animal subjects comprising administering to said human or animal subject an effective therapeutic amount of one or more compounds selected from the group consisting of 2,3-alkylcarbonyloxybenzoic acids and salts thereof wherein the alkylcarbonyl group has 2-18 carbon atoms.

2. The method of claim 1 wherein said 2,3-alkylcarbonyloxybenzoic acid is 2,3-diacetoxybenzoic acid.

3. The method of claim 1 wherein said 2,3-alkylcarbonyloxybenzoic acid is formulated in liquid form in the presence of a buffer.

4. The method of claim 3 wherein said buffer is a sodium bicarbonate buffer such that a sodium salt of said 2,3-alkylcarbonyloxybenzoic acid is formed.

5. The method of claim 1 wherein said 2,3-alkylcarbonyloxybenzoic acid is administered to said subject by means selected from the group consisting of oral, intravenous, topical, cutaneous, transdermal, subcutaneous, intramuscular, inhalation, intranasal, rectal, vaginal, urethral, ocular, sublingual, transpulmonary, intraperitoneal, mucosal, transmucosal, and irrigation administration means.

6. The method of claim 5 wherein said 2,3-alkylcarbonyloxybenzoic acid is administered via nasal or mouth passages.

7. The method of claim 1 wherein said 2,3-alkylcarbonyloxybenzoic acid is administered to said subject in liquid form via intravenous means.

8. The method of claim 1 wherein said 2,3-alkylcarbonyloxybenzoic acid is administered to said subject together with one or more therapeutic agents.

9. The method of claim 8 wherein said one or more therapeutic agents comprises an antibiotic selected from the group comprising fluoroquinolines, doxycycline, rifampin, vancomycin, imipenem, chloramphenicol, penicillin, clindamycin, clarithromycin, gentamicin, beta-lactam antibiotics, ketolides, peptide antibiotics, quinupristin/dalfopristin, linezolid and analogs, homologs, and derivatives thereof which have antibiotic functionality.

10. The method of claim 8 wherein said one or more therapeutic agents comprises at least one antiviral agent selected from the group comprising ribavirin, nucleoside analogues, nonnucleoside inhibitors, protease inhibitors and fusion inhibitors.

11. The method of claim 8 wherein said one or more therapeutic agents comprises at least one antifungal agent selected from the group comprising ketoconazole, fluconazole and itraconazole.

12. The method of claim 1 wherein said 2,3-alkylcarbonyloxybenzoic acid is administered to said subject together with at least one therapeutic agent for the treatment of sepsis.

13. The method of claim 12 wherein said therapeutic agent for the treatment of sepsis is drotrecogin alfa (activated).

14. The method of claim 1 wherein said 2,3-alkylcarbonyloxybenzoic acid is administered to said subject together with at least one compound selected from the group consisting of corticosteroids, mineralosteroids, non-steroidal anti-inflammatory drugs, beta-agonists including dopamine and immunomodulators such as colchicine and macrolides, prolastin, penicillamine, desferroxamine, and vitamins, and analogues of all of the above.

15. The method of claim 1 wherein said 2,3-alkylcarbonyloxybenzoic acid is administered to said subject together with at least one compound selected from the group consisting of therapeutic agents for the prevention and treatment of blood clots, strokes, and myocardial infarction.

16. The method of claim 15 wherein said therapeutic agents are selected from the group consisting of thrombolytic agents, tissue plasminogen activators, and platelet inhibitors.

17. The method of claim 16 wherein said therapeutic agents are selected from the group consisting of alteplase, tenecteplase, anistreplase, reteplase, streptokinase, urokinase, dipyridamole, and clopidogrel.

18. The use in an effective therapeutic amount of one or more compounds selected from the group consisting of 2,3-alkylcarbonyloxybenzoic acids and salts thereof in which the alkylcarbonyl group has 2-18 carbon atoms in the prevention and/or treatment of one or more diseases or conditions selected from the group of diseases and conditions consisting of acute myocardial syndrome, myocardial infarction, ischemic stroke, reperfusion following surgery, gastric mucosal injury (ulcer), hepatic microcirculatory dysfunction (hepatitis and alcoholic liver disease), systemic shock of all types, pancreatic microcirculation disturbances in diabetes, ischemia/reperfusion injury of skeletal muscle caused by thromboembolic events or trauma, ischemia/reperfusion crisis in organ transplant recipients, Duchene's muscular dystrophy, prevention of paraplegia secondary to spinal cord injury, cerebrovascular insufficiency, atherosclerosis, vascular ophthalmopathies, sinusitis, cystic fibrosis, rhinitis, decubitus ulcer, peripheral vascular insufficiency, renal insufficiency, ischemic bowel disease, asthma, chronic obstructive pulmonary disease, pneumonia, bronchitis, pulmonary colonization with bacteria, such as Haemophilus influenzae and Pseudomonas aeruginosa, acne, rosacea, alpha one-antitrypsin deficiency, pulmonary edema, pneumonic plague, congestive heart failure, pulmonary hypertension, lymphangitis, arthritis, bums, diverticulitis, diverticuloisis, lupus erythematosus, rheumatoid arthritis, ankylosing spondylitis, pemphigus vulgaris, pemphigoid, Stevens-Johnson syndrome, drug-induced skin reactions, psoriasis, Alzheimer's disease, multiple sclerosis, eclampsia, pre-eclampsia, malignant hypertension, hypertensive crisis, encephalopathy, encephalitis, meningitis, neuritis, prion-related diseases, systemic hypertension, inflammatory bowel disease, cirrhosis, hepatic encephalopathy, inhalation lung injury, infant respiratory distress syndrome, acute respiratory distress syndrome, severe acute respiratory syndrome, aphtous stomatitis, stomatitis, esophagitis, duodenitis, adenitis, salivary gland inflammation, gingivitis, periodontitis, caries, vaginitis, Parkinson's disease, Huntington's disease, hepatitis, AIDS, Lyme's disease, Rickettsiosis, sarcoidosis, idiopathic pulmonary fibrosis, interstitial lung disease, emphysema, bronchiestasis, a typical mycobacteria, fungal infections, viral infections such as coronavirus, respiratory synctyial virus, metapneumovirus, rhinoviruses, paramyxoviruses, herpes, adenovirus, Epstein Barr virus, parainfluenza viruses, and human immunodeficiency virus, bacterial infections such as Mycoplasma pneumoniae, Chlamydia pneumoniae, Streptococcus pneumoniae, Klebsiella pneumoniae, Staphylococcus aureus, Acinetobacter, Streptococci, Enterococci, Eschericha coli, Mycobacterium tuberculosis and Mycobacterium avium, lichen planus, conjunctivitis, conjunctivitis sicca, nickel, lead, cobalt and other heavy metal poisoning, including accumulation of trace elements and hemochromatosis.

19. The use of claim 18 in the prevention and/or treatment of one or more diseases, conditions, or dysfunctions selected from the group of diseases, conditions and dysfunctions caused by pneumonia, coronavirus, multiple transfusions, trauma, ischemic-reperfusion dysfunctions, stroke, drug overdose and severe acute respiratory syndrome.

20. The use of claim 18 wherein said 2,3-alkylcarbonyloxybenzoic acid is 2,3-diacetoxybenzoic acid.

21. The use of claim 18 wherein said effective therapeutic amount is an amount of from about 0.1 mg. to about 100 mg. per kg. of body weight of said subject.

22. The use of claim 21 wherein said effective therapeutic amount is an amount of from about 5 mg. to about 50 mg. per kg. of body weight of said subject.

23. The use of claim 22 wherein said effective therapeutic amount is an amount of from about 5 mg. to about 20 mg. per kg. of body weight of said subject.

24. The use in an effective therapeutic amount of one or more compounds selected from the group consisting of 2,3-alkylcarbonyloxybenzoic acids and salts thereof in which the alkylcarbonyl group has 2-18 carbon atoms, administered with an effective therapeutic amount of one or more antibiotics.

25. The use of claim 24 wherein said antibiotics are selected from the group comprising fluoroquinolines, doxycycline, rifampin, vancomycin, imipenem, chloramphenicol, penicillin, clindamycin, clarithromycin, gentamicin, beta-lactam antibiotics, ketolides, peptide antibiotics, quinupristin/dalfopristin, linezolid and analogs, homologs, and derivatives thereof which have antibiotic functionality.

26. The use in an effective therapeutic amount of one or more compounds selected from the group consisting of 2,3-alkylcarbonyloxybenzoic acids and salts thereof in which the alkylcarbonyl group has 2-18 carbon atoms is administered together with an effective therapeutic amount of one or more antiviral agents selected from the group comprising ribavirin, nucleoside analogues, nonnuceloside inhibitors, protease inhibitors, and fusion inhibitors.

27. The use in an effective therapeutic amount of one or more compounds selected from the group consisting of 2,3-alkylcarbonyloxybenzoic acids and salts thereof in which the alkylcarbonyl group has 2-18 carbon atoms is administered together with an effective therapeutic amount of one or more antifungal agents selected from the group comprising ketoconazole, fluconazole and itraconazole.

28. The use in an effective therapeutic amount of one or more compounds selected from the group consisting of 2,3-alkylcarbonyloxybenzoic acids and salts thereof in which the alkylcarbonyl group has 2-18 carbon atoms combined with an effective therapeutic amount of at least one therapeutic agent for the treatment of sepsis.

29. The use of claim 28 wherein said therapeutic agent for the treatment of sepsis is drotrecogin alfa (activated).

30. The use in an effective therapeutic amount of one or more compounds selected from the group consisting of 2,3-alkylcarbonyloxybenzoic acids and salts thereof in which the alkylcarbonyl group has 2-18 carbon atoms combined with an effective therapeutic amount of one or more therapeutic agents selected from the group consisting of corticosteroids, mineralosteroids, non-steroidal anti-inflammatory drugs, beta-agonists including dopamine, immunomodulators such as colchicine and macrolides, prolastin, penicillamine, desferroxamine, and vitamins, and analogues of all of the above.

31. The use in an effective therapeutic amount of one or more compounds selected from the group consisting of 2,3-alkylcarbonyloxybenzoic acids and salts thereof in which the alkylcarbonyl group has 2-18 carbon atoms combined with one or more therapeutic agents selected from the group consisting of therapeutic agents for the prevention and treatment of blood clots, strokes, and myocardial infarction.

32. The use of claim 31 wherein said therapeutic agents are selected from the group consisting of thrombolytic agents, tissue plasminogen activators, and platelet inhibitors.

33. The use of claim 32 wherein said therapeutic agents are selected from the group consisting of alteplase, tenecteplase, anistreplase, reteplase, streptokinase, urokinase, dipyridamole, and clopidogrel.
